(19) United States
(12) Patent Application Publication
(10) Pub. No.: US 2026/0245365 A1
(43) Pub. Date: Aug. 20, 2026

(54) PROFICIENCY EVALUATION METHOD, PROFICIENCY EVALUATION DEVICE, AND TRAINED MODEL

(71) Applicant: The University of Osaka, OSAKA (JP)

(72) Inventors: Ryo KAWASAKI, Osaka (JP); Liangzhi LI, Osaka (JP); Yuta NAKASHIMA, Osaka (JP); Hajime NAGAHARA, Osaka (JP); Kohji NISHIDA, Osaka (JP)

(21) Appl. No.: 19/160,655

(22) PCT Filed: Mar. 5, 2024
(86) PCT No.: PCT/JP2024/008200

§ 371 Date: Aug. 28, 2025

(30) Foreign Application Priority Data
Mar. 7, 2023 (JP) 2023-034748

(51) Int. Cl. G06V 20/40; G16H 30/20

(52) U.S. Cl. CPC G06V 20/41; G16H 30/20; G06V 2201/031; G06V 2201/034

(57) ABSTRACT

A proficiency evaluation method includes: acquiring a surgery video and extracting an evaluation target video; detecting a surgical instrument from the evaluation target video; calculating a tip index related to a tip portion of a surgical functional element of the surgical instrument and a proximal end index related to the proximal end portion of the surgical functional element; and evaluating the operative attainment level based on the tip index, the proximal end index, and an operation time of the surgical instrument.

### TECHNICAL FIELD

The present invention relates to a proficiency evaluation method, a proficiency evaluation device, and a trained model that can be used to evaluate the skill attainment levels of doctors in ophthalmic surgery.

### BACKGROUND ART

Traditionally, proficient instructing doctors have visually observed surgical procedures to subjectively evaluate the quality of the surgical procedures. In recent years, there has been increasing social demand for objective evaluations in ophthalmic surgery to clearly indicate whether a surgeon with a certain level of skill will be performing the surgery.

Non-Patent Literature (NPL) 1 discloses a technique of distinguishing proficient surgeons from novice ones by performing, during the anterior capsule incision step in cataract surgery, deep learning of surgery videos using temporal convolutional neural networks (TCNs) to detect instruments and using the speed of movement of the tips of the surgical instruments (instrument tip velocities) and the optical flow, which is the vector of the object's motion. In addition, it has been reported that the accuracy and area under the receiver operating characteristics curve (AUC) were used as evaluation indices for the discrimination capability, and the values were 0.848 and 0.863 for the speed of movement of the tip of the surgical instrument (instrument tip velocities) and 0.634 and 0.803 for the optical flow, respectively.

NPL 2 and 3 have reported that, in the anterior capsule incision step in cataract surgery, a statistically significant difference was found between proficient surgeons and novice surgeons in the total movement distance (path length), the number of movements, and the operation time (time taken) of surgical instruments using the Kanade-Lucas-Tomasi tracking algorithm for surgery videos.

NPL 4 discloses a technique in which, in the anterior capsule incision step in cataract surgery, a deep learning model based on Inception v3 has been created using the development of surgical complications as teacher labels to calculate indices suggesting the probability of the development of a surgical complication as an integrated risk indicator (IRI). In addition, it has been reported that statistically significant differences in IRI and procedure completion time between instructing doctors and residents were recognized.

### PRIOR ART DOCUMENTS

NPL 1: Tae Soo Kim et al., “Objective assessment of intraoperative technical skill in capsulorhexis using videos of cataract surgery”, International Journal of Computer Assisted Radiology and Surgery 2019, Vol. 14, 1097 to 1105.
NPL 2: Shafi Balal et al., “Computer analysis of individual cataract surgery segments in the operating room”, Eye 2019, Vol. 33, 313 to 319.
NPL 3: Phillip Smith et al., “Phaco Tracking”, An Evolving Paradigm in Ophthalmic Surgical Training, JAMA OPHTHALMOL 2013, Vol. 131, 659 to 661.
NPL 4: Hitoshi Tabuchi et al., “Real-time artificial intelligence evaluation of cataract surgery: A preliminary study on demonstration experiment”, Taiwan J Ophthalmol 2022, Vol. 12, 147 to 154.

### DISCLOSURE OF THE INVENTION

### Problem to be Solved by the Invention

Meanwhile, amidst the decreasing number of upcoming surgeons, it is necessary to ascertain the proficiency based on an objective index to promote surgical techniques to be learned more efficiently, and to utilize the proficiency for education and guidance. Nowadays, the development of artificial intelligence technology has enabled videos to be analyzed and indexed, which was difficult in the past.

In NPL 1, although proficient surgeons and novice surgeons can be distinguished based on the moving speed of the tip of a surgical instrument and the optical flow, there is room for further improvement in evaluation accuracy. In NPL 2 to NPL 4, although it was reported that a statistically significant difference has been recognized between proficient surgeons and novice surgeons, an index that is difficult to quantify in the field of ophthalmic surgery (for example, cataract surgery) is not automatically calculated to evaluate the proficiency.

Therefore, a proficiency evaluation method, a proficiency evaluation device, and a trained model that improve the evaluation accuracy in the skill attainment level of doctors in ophthalmic surgery are desired.

### Means for Solving the Problem

According to an aspect of the present invention, a proficiency evaluation method of evaluating an operative attainment level of a doctor using a surgical instrument includes: acquiring a surgery video and extracting an evaluation target video; detecting the surgical instrument from the evaluation target video; calculating a tip index related to a tip portion of a surgical functional element of the surgical instrument and a proximal end index related to a proximal end portion of the surgical functional element; and evaluating the operative attainment level based on the tip index, the proximal end index, and an operation time of the surgical instrument. In addition, according to an aspect of the present invention, a proficiency evaluation device for evaluating an operative attainment level of a doctor using a surgical instrument includes: an extraction unit that acquires a surgery video and extracts an evaluation target video; a detection unit that detects the surgical instrument from the evaluation target video; an index calculation unit that calculates a tip index related to a tip portion of a surgical functional element of the surgical instrument and a proximal end index related to a proximal end portion of the surgical functional element; and an evaluation unit that evaluates the operative attainment level based on the tip index, the proximal end index, and an operation time of the surgical instrument. In addition, the proficiency evaluation device may further include a display unit that displays an evaluation result from the evaluation unit.

In the present method or the present device, the skill attainment level of a doctor in ophthalmic surgery can be determined by evaluating the operative attainment level of the doctor using a surgical instrument based on a tip index related to the tip portion of a surgical functional element of the surgical instrument, the proximal end index related to the proximal end portion of the surgical functional element, and an operation time of the surgical instrument. Moreover, the present method or the present device can determine the skill attainment level with higher accuracy than existing models (for example, NPL 1). In particular, the proximal end index is an important index that serves as a fulcrum in the operation step of the surgical instrument, and contributes to improvement of evaluation accuracy.

In addition, with respect to the proficiency in surgery, the skill attainment level is evaluated only by acquiring a surgery video, rather than being subjectively evaluated by an instructing doctor who visually observed the procedures of surgery to determine the skillfulness, and thus the surgical skill evaluation can be presented automatically as an objective index. As a result, this configuration can be applied to presenting the attainment level in surgical education and indicating to patients that the surgeon is sufficiently proficient in that skill.

In this way, the proficiency evaluation method and the proficiency evaluation device for improving the evaluation accuracy in the skill attainment level of doctors in ophthalmic surgery can be achieved.

In another aspect, the surgery video is a video related to cataract surgery, the tip index includes a sum value of a distance difference from a pupil center position to the tip portion between preceding and succeeding frames of the evaluation target video, and the proximal end index includes a sum value of a distance difference from the pupil center position to the proximal end portion between preceding and succeeding frames of the evaluation target video.

With this configuration, if a surgery video is a video related to cataract surgery, many videos can be obtained to improve the evaluation accuracy. In addition, when the tip index and the proximal end index are the distance difference from the pupil center position to the tip portion between preceding and succeeding frames of the evaluation target video, the amount of calculation can be reduced, and a large amount of proficiency data can be accumulated.

In another aspect, the tip index and the proximal end index include a distribution of the pupil center positions in all frames of the evaluation target video.

If a distribution of pupil center positions is included as described above, it is possible to fairly evaluate the proficiency of a doctor who has performed the surgery for the patient with a large amount of pupil movement during the cataract surgery.

Another aspect is a position at which the surgical functional element and an eye surface are in contact with each other.

In this way, if the position at which the surgical functional element and the eye surface are in contact with each other is set as the proximal end portion, it is easier for skill differences to emerge, leading to improvement in the evaluation accuracy.

An aspect of the present invention is a trained model that functions via a computer, the trained model: receiving an input of a surgery video; and outputting a score based on a tip index related to a tip portion of a surgical functional element of a surgical instrument, a proximal end index related to a proximal end portion of the surgical functional element, and an operation time of the surgical instrument.

With the trained model as in this configuration, the skill attainment level of the doctor in ophthalmic surgery can be determined. Moreover, in the present configuration, the skill attainment level can be determined with higher accuracy than in an existing model (for example, NPL 1). Furthermore, since the skill attainment level is evaluated only by acquiring a surgery video, the skill evaluation for the surgery can be presented automatically and as an objective index. In this way, the trained model that improves the evaluation accuracy in the skill attainment level of doctors in ophthalmic surgery can be achieved.

In another aspect, an evaluation target video extracted from the surgery video is input.

If a surgery video is processed into an evaluation target video for input, without being input as it is as described in the present configuration, noise is reduced, and the evaluation accuracy can be further improved.

In another aspect, the surgery video is a video related to cataract surgery, the tip index includes a sum value of a distance difference from a pupil center position to the tip portion between preceding and succeeding frames of the evaluation target video, and the proximal end index includes a sum value of a distance difference from the pupil center position to the proximal end portion between preceding and succeeding frames of the evaluation target video.

With this configuration, if a surgery video is a video related to cataract surgery, many videos can be obtained to improve the evaluation accuracy. In addition, when the tip index and the proximal end index are the distance difference from the pupil center position to the tip portion between preceding and succeeding frames of the evaluation target video, the amount of calculation can be reduced, and a large amount of proficiency data can be accumulated.

In another aspect, the tip index and the proximal end index include a distribution of the pupil center positions in all frames of the evaluation target video.

If a distribution of pupil center positions is included as described above, it is possible to fairly evaluate the proficiency of a doctor who has performed surgery for a patient with a large amount of pupil movement during the cataract surgery.

Another aspect is a position at which the surgical functional element and an eye surface are in contact with each other.

In this way, if the position at which the surgical functional element and the eye surface are in contact with each other is set as the proximal end portion, it is easier for skill differences to emerge, leading to improvement in the evaluation accuracy.

### BRIEF DESCRIPTION OF DRAWINGS

FIG. 1 is a diagram illustrating an entire system for realizing a proficiency evaluation method according to the present embodiment.

FIG. 2 is a block diagram of a proficiency evaluation device according to the present embodiment.

FIG. 3 is a flowchart for realizing the proficiency evaluation method according to the present embodiment.

FIG. 4 is an explanatory diagram of deep learning related to detection of a surgical instrument according to the present embodiment.

FIG. 5 is an explanatory diagram in which a surgical instrument is detected.

FIG. 6 is an explanatory diagram in which position correction is performed at a pupil center.

FIG. 7 is a diagram illustrating an implementation example of the proficiency evaluation method.

FIG. 8 is a comparison diagram illustrating an example in which the operative attainment level was evaluated.

FIG. 9 is an ROC curve diagram of an example in which the evaluation step was executed using a trained model obtained through machine learning.

FIG. 10 is an example of a screen displaying a proficiency evaluation result.

### DESCRIPTION OF EMBODIMENTS

Hereinafter, embodiments of a proficiency evaluation method, a proficiency evaluation device, and a trained model according to the present invention will be described with reference to the drawings. However, the present invention is not limited to the following embodiments, and various modifications can be made without departing from the gist of the present invention.

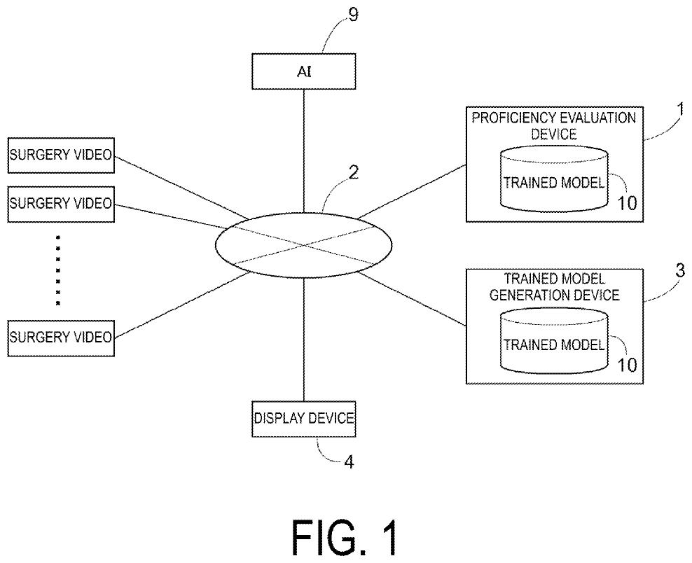

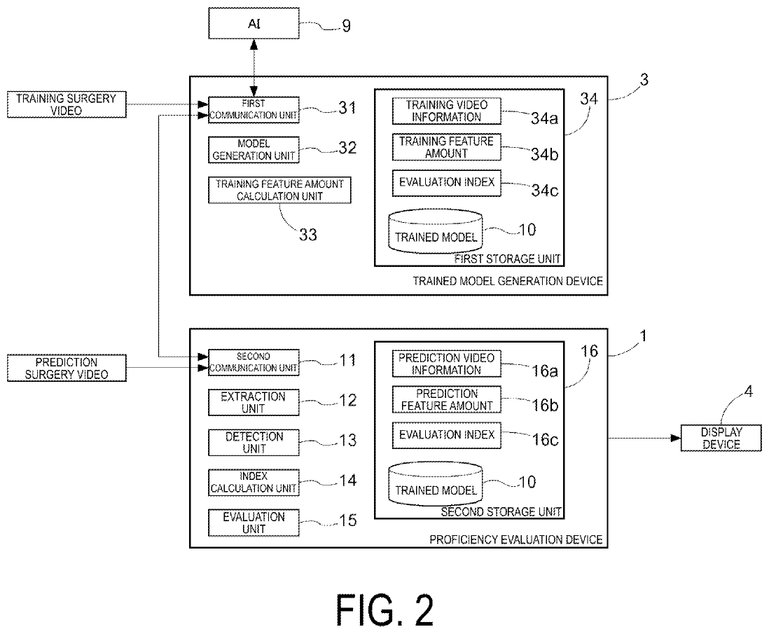

A system configuration used for the proficiency evaluation method will be described with reference to FIGS. 1 and 2. One or a plurality of proficiency evaluation devices 1 usable for evaluating the skill attainment level of a doctor in ophthalmic surgery are connected to an Internet line 2.

Examples of the proficiency evaluation device 1 include a monitor device, an imaging terminal, and the like installed in medical sites (for example, an operating room), a server device including a personal computer and the like installed in a management room, and a mobile terminal. In addition, a trained model generation device 3, a display device 4, and an artificial intelligence 9 (AI) are connected to the Internet line 2. Examples of the display device 4 include a mobile terminal such as a tablet or a smartphone capable of displaying an evaluation result output from the proficiency evaluation device 1. Here, the AI 9 may be provided on the Internet line 2 or may be provided in the trained model generation device 3. In addition, the trained model generation device 3 and the proficiency evaluation device 1 may be the same device, or the display device 4 (an example of a display unit) may be built in the proficiency evaluation device 1, and the respective functions can be used alone or in combination. In addition, the display device 4 may be an imaging device installed in a medical site (for example, an operating room) or a dedicated apparatus in a management room, and can be used as various diagnostic auxiliary apparatuses for evaluating the skill attainment level of a doctor in ophthalmic surgery.

The proficiency evaluation device 1 is an apparatus that automatically evaluates the operative attainment level of a doctor using a surgical instrument when a surgery video is input. A surgery video of the present embodiment includes a video related to cataract surgery. Note that the surgery video may be of a retinal detachment surgery, a vitreous surgery, a surgery using a balloon, or the like.

As illustrated in FIG. 2, the trained model generation device 3 includes a first communication unit 31, a model generation unit 32, a training feature amount calculation unit 33, and a first storage unit 34.

The first communication unit 31 is an interface that transmits and receives data to and from the proficiency evaluation device 1, the AI 9, and the like via the Internet line 2. The first communication unit 31 may receive data directly from the proficiency evaluation device 1, or may accumulate data acquired by the proficiency evaluation device 1 in a server (not illustrated) and receive the accumulated data from the server.

The first storage unit 34 includes a non-transitory storage medium such as an HDD or an SSD or a transitory storage medium such as a RAM, and stores programs or applications executed by a processor. The first storage unit 34 stores training video information 34a acquired via the first communication unit 31. The training video information 34a includes time-series data of images related to cataract surgery, and may be deleted after being converted into a training feature amount 34b described below.

The first storage unit 34 stores the training feature amount 34b calculated by the training feature amount calculation unit 33 for machine learning. The training feature amount 34b is generated by executing deep learning based on the training video information 34a. The training feature amount 34b may be deleted when not used for reinforcement learning.

Deep learning is performed by the AI 9 including a known convolutional neural network. The convolutional neural network constructs a deep hierarchical model that imitates the human neural circuit, and infers the training feature amount 34b. The deep learning is configured by a known application (for example, ResNeSt, TCN, Semantic Segmentation, or the like) provided via the Internet line 2.

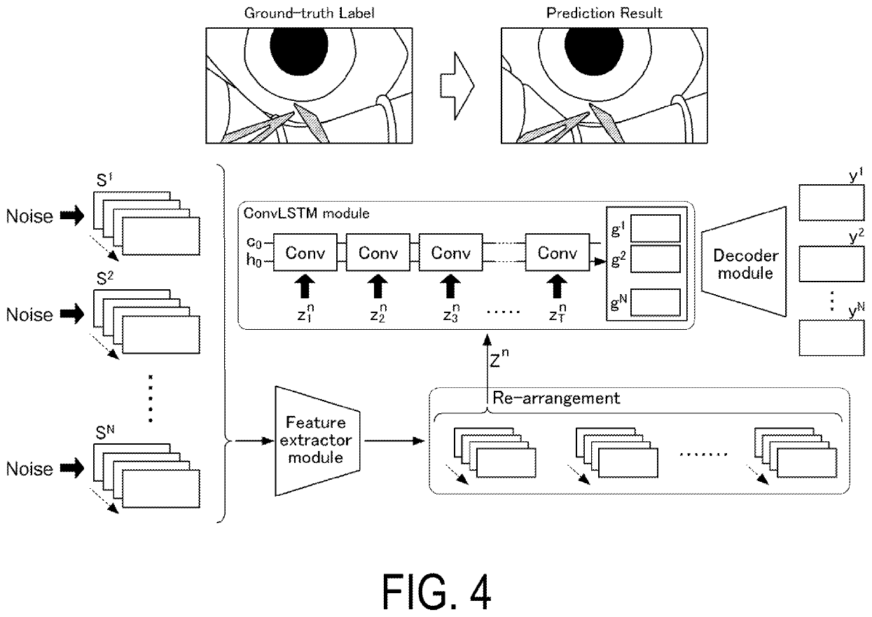

In addition, in a case where a trained model 10 is generated through deep learning, the training feature amount calculation unit 33 is built in the trained model 10. In particular, the grouping and the coupling of the convolution layers are performed by using a ResNeSt (Split-Attention Networks) model using the time-series feature information inside the trained model 10 to compress the amount of data, and the feature amount of the surgical instrument is extracted by using a Semantic Segmentation model to be used as the training feature amount 34b. Here, the time-series feature information is obtained by arranging the training video information 34a in time series and set as a feature amount. FIG. 4 illustrates a conceptual diagram of deep learning. The time-series feature information is rearranged (Re-arrangement) from the training video information 34a by using the ResNeSt model, an annotated video data is generated, data compression is performed by the convolution layer (ConvLSTM module), and the feature amount of the surgical instrument is extracted by the Semantic Segmentation model or the like (Decoder module). As a result, a ground-truth label drawn from the actual video and the prediction result are approximated. Note that the model used for deep learning is not limited to ResNeSt, and may be ResNet, ResNeXt, SE-Net, SK-Net, TCN, or the like.

As illustrated in FIG. 2, the first storage unit 34 stores the trained model 10. The trained model 10 is a model that functions via a computer, and is obtained from deep learning and supervised machine learning.

In the machine learning, variable selection is executed in a logistic regression model using a least absolute shrinkage and selection operator (lasso), and an evaluation index is set. In the present embodiment, a model including, as variables, an instrument tip deviation index (roughness index) in addition to a step end time (total time, an example of an operation time of a surgical instrument), an instrument tip stability level index (tip-stability index, an example of a tip index), and an instrument insertion portion stability index (insertion point-stability index, an example of a proximal end index) is started, variables are in 10-fold cross-validation, and thereby creating a final model. As a result, the instrument tip deviation index (roughness index) is excluded from the variables providing meaningful information, and a model consisting of three variables including the step end time (total time), the instrument tip stability level index (tip-stability index), and the instrument insertion portion stability index (insertion point-stability index), is adopted. Note that the machine learning may be a regression model (for example, ridge) other than lasso, a tree-based model such as a decision tree, or an ensemble model such as XGboost or Random Forest.

The model generation unit 32 includes a processor and generates the trained model 10. The processor includes an ASIC, an FGPA, a CPU, or other hardware for executing an application or the like stored in the first storage unit 34 (the same applies hereinafter).

The training feature amount calculation unit 33 includes a processor, calculates a plurality of training feature amounts 34b from the training video information 34a through the above-described deep learning, analyzes the plurality of calculated training feature amounts 34b, obtains the degree of influence of each feature amount on the evaluation index 34c, and sets a regression coefficient. Note that the training feature amount calculation unit 33 may extract a plurality of (for example, three) indices in descending order of the influence degrees through deep learning, and the model generation unit 32 may use the plurality of extracted evaluation indices as the training feature amounts 34b.

The step end time (total time) is the duration from the start to the end of the anterior capsule incision, which is performed using CCC forceps or a cystotome (hereinafter referred to as “CCC forceps or the like”) as surgical instruments. The instrument insertion portion stability index (insertion point-stability index) indicates the amount of movement of a proximal end portion (the site that is inserted into the cornea and comes in contact with the cornea, i.e. the incision slit) of the CCC forceps or the like. That is, the proximal end portion corresponds to the position at which the surgical functional element of a surgical instrument contacts the eye surface. The instrument tip stability level index (tip-stability index) is the amount of movement of the tip portion (the portion in contact with the anterior capsule of the crystalline lens) of the CCC forceps or the like. Note that the instrument tip deviation index (roughness index) that is not adopted in the present embodiment is the amount of deviation of the tip portion (the portion in contact with the anterior capsule of the crystalline lens) of the CCC forceps or the like from the reference range. Note that the step end time (total time) may be automatically extracted through deep learning (ResNeSt or the like), or may be a measured time obtained by measuring the anterior capsule incision step for each surgery.

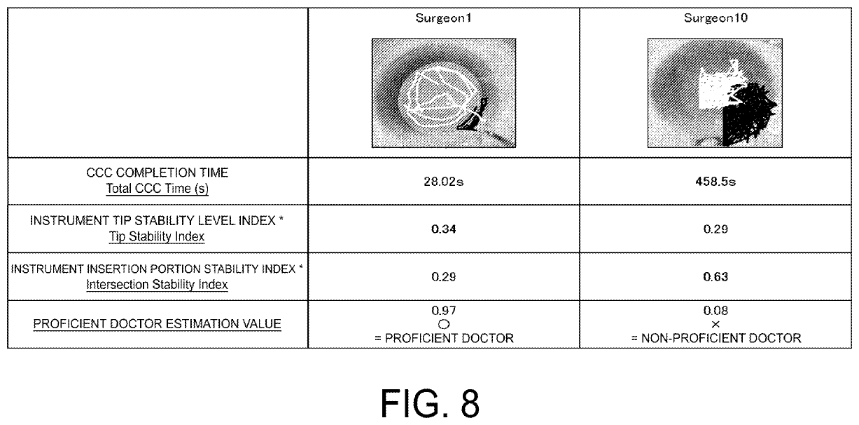

The trained model 10 generated as described above outputs a score of the proficiency. The trained model 10 to which surgery videos are input outputs a score (proficient surgeon estimation value) based on a tip index (instrument tip stability level index) about the tip portion (the portion in contact with the anterior capsule of the crystalline lens) of a surgical functional element (anterior capsule incision element) of a surgical instrument (CCC forceps or the like), a proximal end index (instrument insertion portion stability index) related to a proximal end portion (incision slit) of the surgical functional element (anterior capsule incision element) of the surgical instrument (CCC forceps or the like), and an operation time (step end time) of the surgical instrument. As illustrated in FIG. 8, the trained model 10 outputs a score of 0.97 indicating a proficient doctor in surgery 1 (Surgeon 1) and a score of 0.08 indicating a non-proficient doctor in surgery 10 (Surgeon 10). The anterior capsule incision element refers to an element of the CCC forceps or the like that is positioned between the cornea and the crystalline lens in the anterior capsule incision step.

The proficiency evaluation device 1 includes a second communication unit 11, an extraction unit 12, a detection unit 13, an index calculation unit 14, an evaluation unit 15, and a second storage unit 16 as illustrated in FIG. 2.

The second communication unit 11 is an interface that transmits and receives data to and from the trained model generation device 3, the display device 4, the AI 9, and the like via the Internet line 2. The second communication unit 11 may receive the surgery video data directly from the Internet line 2, or may accumulate the data acquired by the proficiency evaluation device 1 in a server (not illustrated) and receive the accumulated data from the server.

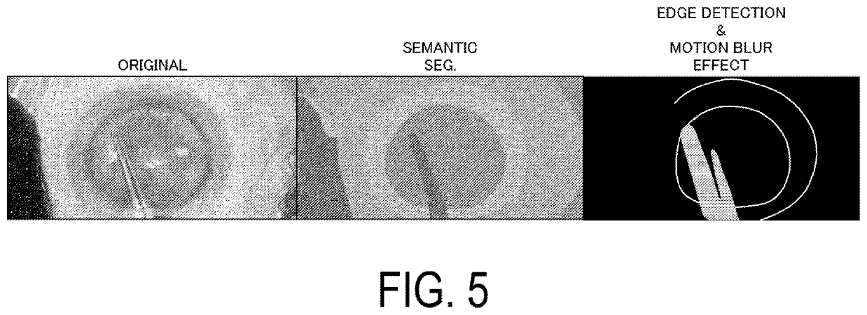

The extraction unit 12 includes a processor, acquires the surgery video for prediction through the second communication unit 11, and extracts the evaluation target video. As illustrated in the left diagram (ORIGINAL) of FIG. 5, the extraction unit 12 extracts a target video (evaluation target video) related to the anterior capsule incision step in the cataract surgery via the AI 9 such as the ResNeSt included in the trained model 10.

The detection unit 13 includes a processor and detects a surgical instrument from the evaluation target video. As illustrated in the right diagram (EDGE DETECTION & MOTION BLUR EFFECT) of FIG. 5, the detection unit 13 detects a surgical instrument (CCC forceps or the like) from the evaluation target video related to the anterior capsule incision step via the AI 9 such as Semantic Segmentation included in the trained model 10. Then, the detection unit 13 detects the positions of the tip portion (the portion in contact with the anterior capsule of the crystalline lens) and the proximal end portion (the incision slit) of the surgical functional element (the anterior capsule incision element) of the surgical instrument (the CCC forceps or the like) as the prediction feature amount 16b.

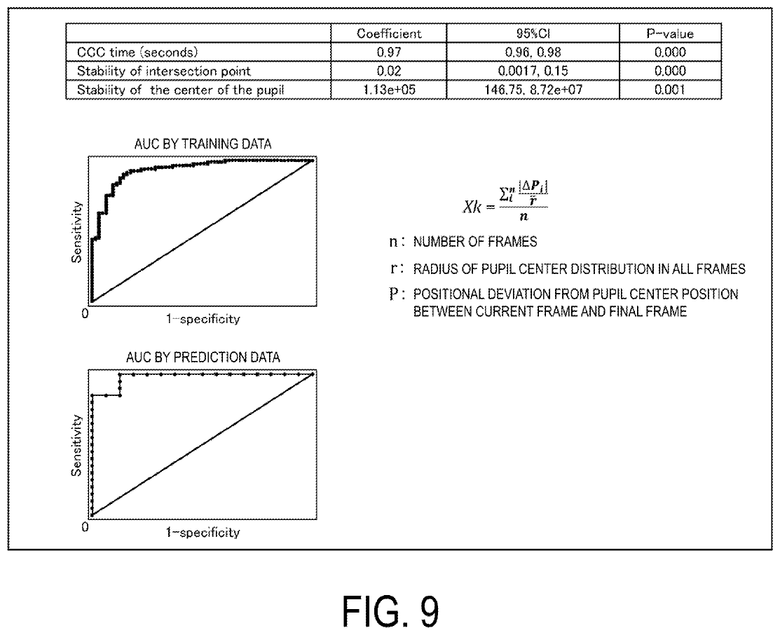

The index calculation unit 14 includes a processor, and calculates a tip index (instrument tip stability level index) about the tip portion (the portion in contact with the anterior capsule of the crystalline lens) of the surgical functional element (anterior capsule incision element) of a surgical instrument (CCC forceps or the like) and a proximal end index (instrument insertion portion stability index) related to the proximal end portion (incision slit) of the surgical functional element (anterior capsule incision element) of the surgical instrument (CCC forceps or the like). The tip index (Xk (k=2)) includes a sum value of the distance difference from the pupil center position to the tip portion between the preceding and succeeding frames of the evaluation target video, and the proximal end index (Xk (k=3)) includes a sum value of the distance difference from the pupil center position to the proximal end portion between the preceding and succeeding frames of the evaluation target video as illustrated in FIG. 9. The tip index and the proximal end index include the distribution of the pupil center positions in all the frames of the evaluation target video. The distribution of the pupil center positions according to the present embodiment is set to the radius of a virtual circle drawn by capturing the pupil center positions in time series. Since the pupil center position in each frame moves during the anterior capsule incision step, the movement amount of the surgical instrument (CCC forceps or the like) can be reflected with high accuracy by dividing the sum value of the distance differences from the pupil center position to the tip portion and the proximal end portion by the radius of the distribution of the pupil center positions.

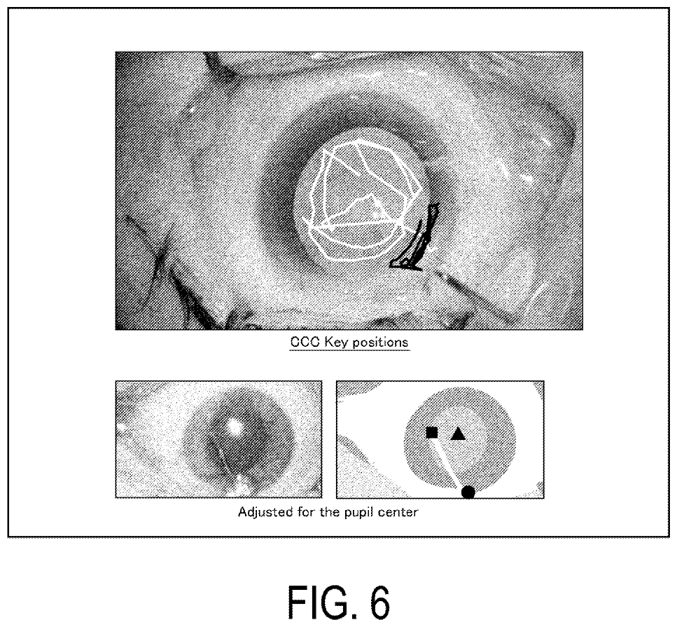

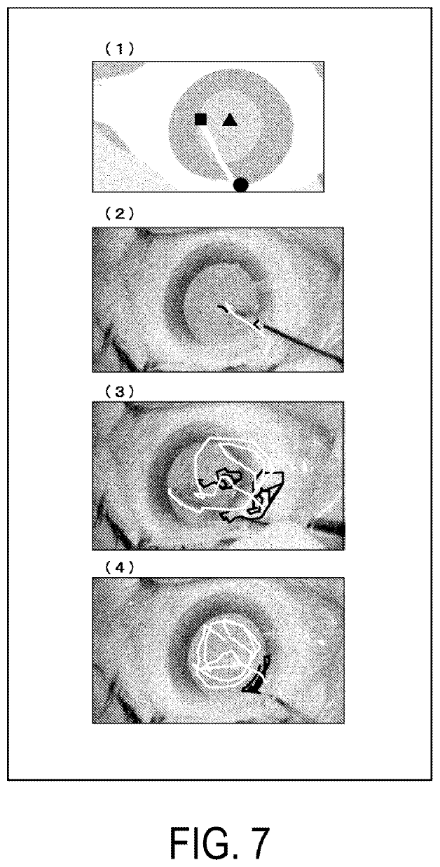

FIG. 7 illustrates the amount of movement of the surgical instrument (CCC forceps or the like). When the pupil center position (the triangular mark in FIG. 7(1)) is not taken into consideration as illustrated in FIG. 7(3), the tip portion (the site in contact with the anterior capsule of the crystalline lens, the square mark in FIG. 7(1), the white line in FIG. 7(3) of the surgical functional element (anterior capsule incision element) and the proximal end portion (the incision slit, the circular mark in FIG. 7(1), the black line in FIG. 7(3)) of the surgical functional element (anterior capsule incision element) vary in proportion to the movement of the pupil center position. On the other hand, when the pupil center position is taken into consideration (when the pupil center is fixed) as illustrated in FIG. 7(4), the tip portion (the site in contact with the anterior capsule of the crystalline lens, the white line in FIG. 7(4)) of the surgical functional element (anterior capsule incision element) and the proximal end portion (the incision slit, the black line in FIG. 7(4)) of the surgical functional element (anterior capsule incision element) can be distinguished with reference to the pupil center position. That is, in the present embodiment, the position of the surgical functional element (anterior capsule incision element) is corrected with reference to the pupil center position (triangle mark) as illustrated in the lower diagram (Adjusted for the pupil center) of FIG. 6.

The evaluation unit 15 includes a processor, and evaluates the operative attainment level based on the tip index (instrument tip stability level index) and the proximal end index (instrument insertion portion stability index) calculated by the index calculation unit 14 and the operation time (step end time) of the surgical instrument (CCC forceps or the like). Specifically, the respective regression coefficients (Coefficient) illustrated in FIG. 9 are multiplied by the tip index (instrument tip stability level index) and the proximal end index (instrument insertion portion stability index) calculated by the index calculation unit 14 and the operation time (step end time (total time)) of the surgical instrument (CCC forceps or the like), and the operative attainment level (proficient doctor estimation value) is output as a score as illustrated in FIG. 8 through a mathematical model using the three indices. Then, if the score exceeds a threshold value (for example, 0.5), the doctor is evaluated as a proficient doctor, and if the score is equal to or lower than the threshold value (for example, 0.5), the doctor is evaluated as a non-proficient doctor. Note that the evaluation unit 15 may evaluate doctors in multiple stages, such as evaluating a doctor as a highly proficient doctor when the score exceeds a first threshold value (for example, 0.8), as an intermediate-proficient doctor when the score exceeds a second threshold value (for example, 0.6) and is equal to or lower than the first threshold value (for example, 0.8), as a low-proficient doctor when the score exceeds a third threshold value (for example, 0.4) and is equal to or lower than the second threshold value (for example, 0.6), and as a non-proficient doctor when the score is equal to or lower than the third threshold value (for example, 0.4).

In the present embodiment, the following formula (1) is used as a mathematical model using three the indices. Here, a is a constant term, X1 is a step end time, a is a regression coefficient of the step end time, X2 is a tip index (instrument tip stability level index), b is a regression coefficient of the tip index (instrument tip stability level index), X3 is a proximal end index (instrument insertion portion stability index), and c is a regression coefficient of the proximal end index (instrument insertion portion stability index)

$$p = Pr ( Y = 1 \vert X ) = \frac{1}{1 + {e}^{- ( α + a * {X}_{1} + b * {X}_{2} + c * {X}_{3} )}} \qquad [ Math . 1 ]$$

The second storage unit 16 is configured by a non-transitory storage medium such as an HDD or an SSD or a transitory storage medium such as a RAM, and stores programs or applications executed by a processor, as illustrated in FIG. 2. The second storage unit 16 stores prediction video information 16a acquired via the second communication unit 11 and the trained model 10 generated by the trained model generation device 3. The prediction video information 16a includes time-series data of images related to cataract surgery, and may be deleted after being converted into the prediction feature amount 16b to be described below. In addition, the prediction feature amount 16b may be deleted when not used for reinforcement learning.

The second storage unit 16 stores the prediction feature amount 16b calculated by the detection unit 13 to input the prediction feature amount to the trained model 10 machine-learned by the trained model generation device 3. As described above, the prediction feature amount 16b is detected by inputting a prediction video to the trained model 10.

The second storage unit 16 stores an evaluation result 16c output from the trained model 10.

The evaluation result 16c is created for each doctor. The evaluation result 16c includes a receiver operating characteristic (ROC) curve indicating a prediction accuracy of the trained model 10. The evaluation result 16c also includes a time-series area under the curve (AUC) calculated from the ROC curve.

The evaluation result 16c stored in the second storage unit 16 is transmitted to the display device 4 via the second communication unit 11. By displaying the evaluation result 16c on the display device 4, the result can be applied, such as presenting the attainment level in surgical education, and indicating to patients that the surgeons are sufficiently proficient in that skill.

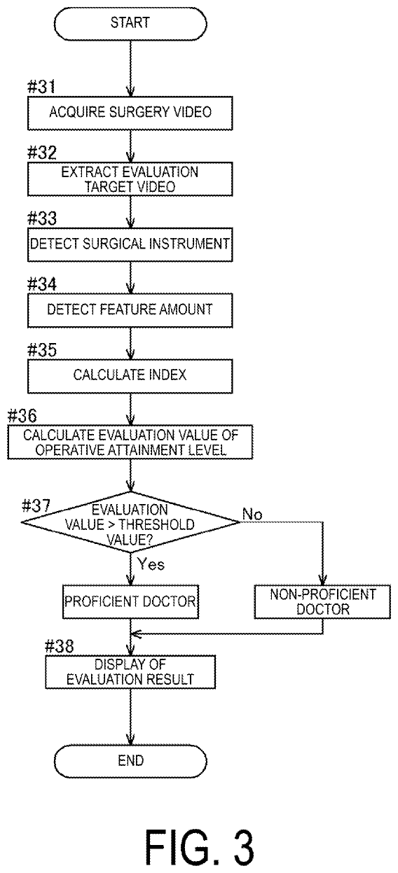

Next, an example of a proficiency evaluation method (program) executed by a computer to evaluate the skill attainment level of a doctor in ophthalmic surgery by using the trained model 10 according to the present embodiment will be described with reference to FIGS. 3 to 9. The proficiency evaluation method includes an extraction step of acquiring a surgery video and extracting an evaluation target video, a detection step of detecting a surgical instrument from the evaluation target video, an index calculation step of calculating a tip index related to a tip portion of a surgical functional element of the surgical instrument and a proximal end index related to the proximal end portion of the surgical functional element, and an evaluation step of evaluating the operative attainment level based on the tip index, the proximal end index, and an operation time of the surgical instrument.

The trained model generation device 3 acquires the training video information 34a over a predetermined period via the Internet line 2. Next, the model generation unit 32 of the trained model generation device 3 executes deep learning including a convolutional neural network using the training video information 34a (cataract surgery video) for input data to generate the trained model 10 (see FIG. 4). The trained model 10 includes a ResNeSt model, a semantic segmentation model, and the evaluation index 34c suitable for a cataract surgery video.

Next, the proficiency evaluation device 1 acquires the trained model 10 from the trained model generation device 3 via the second communication unit 11, and acquires the prediction surgery video from the Internet line 2 (#31 in FIG. 3). Next, the extraction unit 12 extracts an evaluation target video (prediction video information 16a) related to the anterior capsule incision step of the cataract surgery from the prediction surgery video via the AI 9 such as ResNeSt included in the trained model 10 (#32 in FIG. 3, extraction step). Next, the detection unit 13 detects a surgical instrument (CCC forceps or the like) from the target video related to the anterior capsule incision step via the AI 9 such as Semantic Segmentation included in the trained model 10 (#33 in FIG. 3, detection step, see the left diagram in FIG. 5 and FIG. 7(1)). Then, the detection unit 13 detects the positions of the tip portion (the portion in contact with the anterior capsule of the crystalline lens) and the proximal end portion (the incision slit) of the surgical functional element (the anterior capsule incision element) of the surgical instrument (the CCC forceps or the like) as the prediction feature amount 16b (#34 in FIG. 3, detection step, see the right diagram in FIG. 5 and FIGS. 7(2) and (3)).

Next, the index calculation unit 14 calculates a tip index (instrument tip stability level index) related to the tip portion (the portion in contact with the anterior capsule of the crystalline lens) of the surgical functional element (anterior capsule incision element) of the surgical instrument (CCC forceps or the like) and the proximal end index (instrument insertion portion stability index) related to the proximal end portion (incision slit) of the surgical functional element (anterior capsule incision element) of the surgical instrument (CCC forceps or the like) (index calculation step, #35 in FIG. 3, see FIG. 6, FIG. 7(4), and FIG. 9). These indices are incorporated in the trained model 10.

Next, the evaluation unit 15 evaluates the operative attainment level based on the tip index (instrument tip stability level index) and the proximal end index (instrument insertion portion stability index) calculated by the index calculation unit 14 and the operation time (step end time) of the surgical instrument (CCC forceps or the like) (evaluation step, #36 in FIG. 3, see FIG. 8). In the example illustrated in FIG. 8, the evaluation unit 15 evaluates the doctor who has performed the surgery 1 (Surgeon 1) whose proficient doctor estimation value is 0.97 as a proficient doctor because the value exceeds the threshold value (0.5) (Yes in #37 in FIG. 3). On the other hand, the evaluation unit 15 evaluates the doctor who has performed the surgery 10 (Surgeon 10) whose proficient doctor estimation value is 0.08 as a non-proficient doctor because the value is equal to or lower than the threshold value (0.5) (No in #37 in FIG. 3). Then, displaying the evaluation result 16c on the display device 4 can be applied to presenting the attainment level of the doctor in surgical education, and indicating to patients that the surgeons are sufficiently proficient in that skill (#38 in FIG. 3). Note that, among the indices illustrated in FIG. 8, the additional indices (instrument tip stability level index, instrument insertion portion stability index) of * are calculated based on the calculation formula illustrated in FIG. 9.

In this way, the skill attainment level of doctors in ophthalmic surgery can be determined by evaluating the operative attainment level of the doctors using the surgical instrument based on the tip index related to the tip portion of the surgical functional element of the surgical instrument, the proximal end index related to the proximal end portion of the surgical functional element, and the operation time of the surgical instrument. Moreover, the present method or the present device can determine the skill attainment level with higher accuracy than existing models (for example, NPL 1). In particular, the proximal end index is an important index that serves as a fulcrum in the operation step of the surgical instrument, and contributes to improvement of evaluation accuracy.

In addition, with respect to the proficiency in surgery, the skill attainment level is evaluated only by acquiring a surgery video, rather than being subjectively evaluated by an instructing doctor who visually observed the procedures of surgery to determine the skillfulness, and thus the surgical skill evaluation can be presented automatically as an objective index. As a result, this configuration can be applied to presenting the attainment level in surgical education and indicating to each patient that the surgeon is sufficiently skilled.

Furthermore, if a surgery video is a video related to cataract surgery, many videos can be obtained to improve the evaluation accuracy. In addition, when the tip index and the proximal end index are the distance difference from the pupil center position to the tip portion between the preceding and succeeding frames of the evaluation target video as in the present embodiment, the amount of calculation can be reduced, and a large amount of proficiency data can be accumulated. Furthermore, since the tip index and the proximal end index include the distribution of pupil center positions, it is possible to fairly evaluate the proficiency of the doctor who has performed the surgery for the patient with a large amount of pupil movement during the cataract surgery.

FIG. 9 illustrates performance evaluation of the trained model 10 according to the present embodiment. In the mathematical model built in the trained model 10, a data set including 100 surgical operations performed by 5 proficient doctors and 79 surgical operations performed by 5 specialist doctors was used to select variables by using a logistic regression model using a least absolute shrinkage and selection operator (lasso). Lasso is a machine learning method, and 143 cases corresponding to 80% of the data were used as a training data set for model creation, and 36 cases corresponding to 20% were used for verification (prediction data set). A model including, as variables, an instrument tip deviation index (roughness index) in addition to a step end time (total time), an instrument tip stability level index (tip-stability index), and an instrument insertion portion stability index (insertion point-stability index) was started, variables were selected in 10-fold cross-validation, and thereby a final model was created. As a result, the instrument tip deviation index (roughness index) was excluded from the variables providing meaningful information, and a model consisting of three variables including the step end time (total time), the instrument tip stability level index (tip-stability index), and the instrument insertion portion stability index (insertion point-stability index) was adopted. The accuracy and the area under the receiver operating characteristics curve (AUC) in the training data set used for the model creation were 0.874 and 0.9345 (95% confidence interval: 0.89556 to 0.97348), respectively. In the prediction data set, the accuracy and the area under the receiver operating characteristics curve (AUC) were 0.889 and 0.9813 (95% confidence interval: 0.94911 to 1.00000), respectively, and a result with higher performance than the result of NPL 1 was obtained.

Next, a training model was created by determining regression coefficients using a logistic regression model (lasso) with the variables of the step end time (total time) and the instrument tip stability level index (tip-stability index) for Comparative Example 1, the variables of the step end time (total time) and the instrument insertion portion stability index (insertion point-stability index) for Comparative Example 2, and the variables of the step end time (total time), the instrument tip stability level index (tip-stability index), and the instrument insertion portion stability index (insertion point-stability index) for the present example. Note that, for training data set and prediction data set, the data set consisting of 100 surgical operations performed by 5 proficient doctors and 79 surgical operations performed by 5 specialist doctors described above was used.

As a result, the AUC in the prediction data set was higher in the present example than in Comparative Examples 1 and 2 as illustrated in Table 1, which proves the significance of using the three indices. In addition, since the AUC was slightly lower in the present example than in the case of starting from a model including the instrument tip deviation index (roughness index) in addition to the step end time (total time), the instrument tip stability level index (tip-stability index), and the instrument insertion portion stability index (insertion point-stability index) as variables (95% confidence interval, 0.94911 to 1.00000), it was found that the performance could be further improved by adding other indices at the stage of variable selection.

TABLE 1

| | Indices used | ROC |
|---|---|---|
| Comparative Example 1 | Step end time + instrument tip stability level index | 0.9152 |
| Comparative Example 2 | Step end time + instrument insertion portion stability index | 0.9073 |
| Present Example | Step end time + instrument tip stability level index + | 0.9402 |
| | instrument insertion portion stability index | |

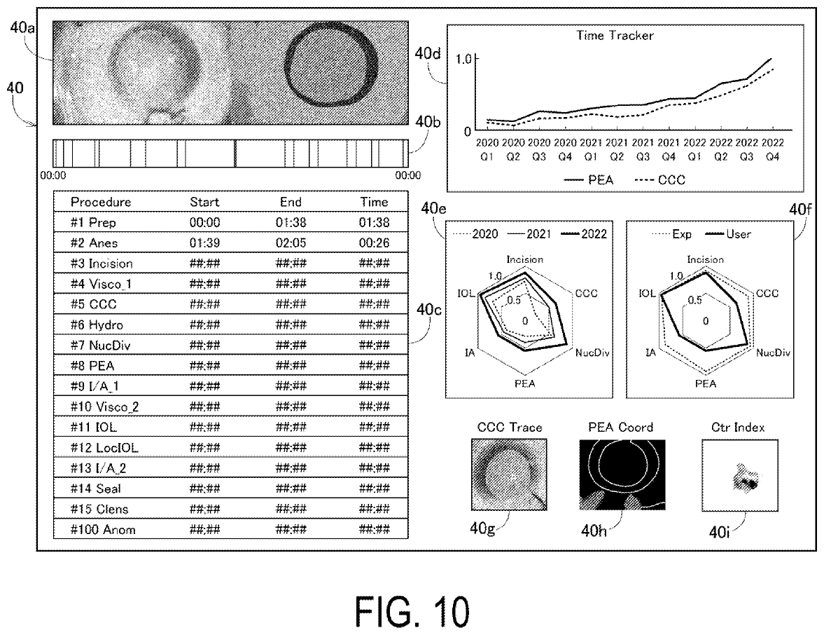

FIG. 10 illustrates a screen 40, which is an example of a screen displaying the proficiency evaluation results output from the proficiency evaluation device 1. The screen 40 is displayed on the display device 4, and also displays a surgery video 40a related to the proficiency evaluation, a graph 40b indicating the time required for the surgery, a table 40c indicating the time and the like required for each step of the surgery, graphs and the like indicating the scores output from the proficiency evaluation device 1. The surgery video 40a may be processed such that the eye tissues are painted in different colors and displayed.

Each step of cataract surgery is classified into, for example, preparing (#1 Prep), anesthetizing (#2 Anes), incising (#3 Incision), injecting Viscoat (viscoelastic material) (#4 Visco_1), anterior capsule incision using CCC forceps or the like (#5 CCC), separating the lens capsule and cortex (Hydro-dissection, #6 Hydro, #7 NucDiv), ultrasound aspiration (#8 PEA), cortex aspiration (#9 I/A_1), injecting Viscoat (viscoelastic material) (#10 Visco_2), inserting an intraocular lens (#11 IOL, #12 LocIOL), aspirating Viscoat (viscoelastic material) (#13 I/A_2), closing the incision (#14 Seal), cleaning (#15 Clens), and others (#100 Anom). The others (#100 Anom) may be a time period during which the above-described steps are not performed or a time period during which a trouble such as a stop of the machine occurs. The graph 40b is a bar graph indicating the time required for each step. The start time, the end time, the required time, and the like of each step are displayed in the table 40c. In addition, the surgery video 40a may be separated for each step, and each step in the graph 40b or the table 40c may be selected to replay the surgery video 40a related to the step.

The proficiency evaluation device 1 in the present embodiment evaluates the proficiency in the anterior capsule incision using the CCC forceps or the like (#5 CCC), but the proficiency evaluation may be performed in the step of the ultrasound aspiration (#8 PEA) or the like, for example in the same manner as the proficiency evaluation related to the anterior capsule incision using the CCC forceps or the like (#5 CCC). The score of each step output by the proficiency evaluation device 1 may be displayed on the table 40c.

The screen 40 may include a graph illustrating a change in the scores of the doctor related to the proficiency evaluation. The change in the scores may be displayed with reference to the proficiency evaluation results of the doctor accumulated in the proficiency evaluation device 1 or the server. The screen 40 displays, for example, a trend 40d of scores related to the anterior capsule incision using the CCC forceps or the like (#5 CCC) and ultrasound aspiration (#8 PEA), which are steps with a high degree of difficulty of improvement in cataract surgery. The trend 40d may be displayed as a mean value of the scores of each year or may be displayed as a mean value of the scores of each quarter. The doctor can figure out his/her own operative attainment level by checking the trend 40d. In addition, the screen 40 may display a radar chart 40e for comparing the scores of the respective steps, or may display a radar chart 40f for comparing the scores with the proficient doctors. This enables the doctors to ascertain the steps with low scores. Note that the trend 40d, the radar chart 40e, and the radar chart 40f may indicate the required time instead of the score. This allows the doctors to ascertain which step takes time and how quickly they are improving.

Furthermore, the screen 40 may display information 40g indicating the amount of movement of the surgical instrument (CCC forceps or the like) related to the surgery video, information 40h indicating the smoothness and tremors of the tip of the surgical instrument, and information 40i indicating whether the pupils have been captured at the center of the screen of the microscope. These pieces of information may be information calculated by the index calculation unit 14. By checking these pieces of information, the doctors can visually determine their proficiency.

### Other Embodiments

(1) Although the proximal end portion of a surgical functional element (anterior capsule incision element) of a surgical instrument (CCC forceps or the like) is described as a position (incision slit) in contact with the eye surface, the proximal end portion of the surgical functional element (anterior capsule incision element) may be set at a position inside (crystalline lens side) or outside the incision slit of the eye. In addition, the proximal end index (instrument insertion portion stability index) may not be corrected at the pupil center position.
(2) Although the trained model 10 is generated through a combination of deep learning using a convolutional neural network and machine learning of logistic regression, the trained model may be generated only through deep learning or only through machine learning.
(3) Although the surgery video is input to the trained model 10, an evaluation target video extracted from the surgery video may be input. The evaluation target video is, for example, a video in which a region of interest related to cataract surgery is extracted in advance. In this way, if a surgery video is processed into an evaluation target video for input, without being input as it is, noise is reduced and the evaluation accuracy can be further improved.
(4) Although the operation time (step end time (total time)), the tip index (instrument tip stability level index (tip-stability index)), and the proximal end index (instrument insertion portion stability index (insertion point-stability index)) of the surgical instrument are exemplified as the evaluation indices selected by the trained model 10, in addition to these, for example, another tip index (instrument tip deviation index (roughness index)) may be used.
(5) Although the tip index (instrument tip stability level index (tip-stability index)) and/or the proximal end index (instrument insertion portion stability index (insertion point-stability index)) are/is set as the sum value of the distance difference from the pupil center position to the tip portion and/or the proximal end portion between the preceding and succeeding frames of the evaluation target video, a different calculation method using an average value of the distance difference may be adopted.

### INDUSTRIAL APPLICABILITY

The present invention can be used for a proficiency evaluation method, a proficiency evaluation device, and a trained model for evaluating the skill attainment level of a doctor in ophthalmic surgery.

### DESCRIPTION OF REFERENCE SIGNS

1: Proficiency evaluation device
4: Display device (display unit)
10: Trained model
12: Extraction unit
13: Detection unit
14: Index calculation unit
15: Evaluation unit

1. A proficiency evaluation method of evaluating an operative attainment level of a doctor using a surgical instrument, the proficiency evaluation method comprising:
acquiring a surgery video and extracting an evaluation target video;
detecting the surgical instrument from the evaluation target video;
calculating a tip index related to a tip portion of a surgical functional element of the surgical instrument and a proximal end index related to a proximal end portion of the surgical functional element; and
evaluating the operative attainment level based on the tip index, the proximal end index, and an operation time of the surgical instrument.

2. The proficiency evaluation method according to claim 1, wherein
the surgery video is a video related to cataract surgery,
the tip index includes a sum value of a distance difference from a pupil center position to the tip portion between preceding and succeeding frames of the evaluation target video, and
the proximal end index includes a sum value of a distance difference from the pupil center position to the proximal end portion between preceding and succeeding frames of the evaluation target video.

3. The proficiency evaluation method according to claim 2, wherein the tip index and the proximal end index include a distribution of the pupil center positions in all frames of the evaluation target video.

4. The proficiency evaluation method according to claim 2, wherein the proximal end portion is a position at which the surgical functional element and an eye surface are in contact with each other.

5. A proficiency evaluation device for evaluating an operative attainment level of a doctor using a surgical instrument, the proficiency evaluation device comprising:
an extraction unit configured to acquire a surgery video and extract an evaluation target video;
a detection unit configured to detect the surgical instrument from the evaluation target video;
an index calculation unit configured to calculate a tip index related to a tip portion of a surgical functional element of the surgical instrument and a proximal end index related to a proximal end portion of the surgical functional element; and
an evaluation unit configured to evaluate the operative attainment level based on the tip index, the proximal end index, and an operation time of the surgical instrument.

6. The proficiency evaluation device according to claim 5, further comprising a display unit configured to display an evaluation result from the evaluation unit.

7. A trained model that functions via a computer, the trained model:
receiving an input of a surgery video; and
outputting a score based on a tip index related to a tip portion of a surgical functional element of a surgical instrument, a proximal end index related to a proximal end portion of the surgical functional element, and an operation time of the surgical instrument.

8. The trained model according to claim 7, wherein an evaluation target video extracted from the surgery video is input.

9. The trained model according to claim 8, wherein
the surgery video is a video related to cataract surgery,
the tip index includes a sum value of a distance difference from a pupil center position to the tip portion between preceding and succeeding frames of the evaluation target video, and
the proximal end index includes a sum value of a distance difference from the pupil center position to the proximal end portion between preceding and succeeding frames of the evaluation target video.

10. The trained model according to claim 9, wherein the tip index and the proximal end index include a distribution of the pupil center positions in all frames of the evaluation target video.

11. The trained model according to claim 9, wherein the proximal end portion is a position at which the surgical functional element and an eye surface are in contact with each other.
